(19) United States
(12) Patent Application Publication
(10) Pub. No.: US 2026/0103650 A1
(43) Pub. Date: Apr. 16, 2026

(54) THERMALLY STABLE COMPOSITIONS AND USE THEREOF AS ANTIFOULANTS

(71) Applicant: ECOLAB USA Inc., St. Paul, MN (US)

(72) Inventors: Jonathan MASERE, Richmond, TX (US); Huan WANG, Shanghai (CN); Pedro Jorge Carvalho CAMPOS, Perre (PT); Satheesh Kumar SAMBANDAM, Richmond, TX (US); Michael David SMITH, Houston, TX (US)

(73) Assignee: ECOLAB USA Inc., St. Paul, MN (US)

(21) Appl. No.: 19/350,955

(22) Filed: Oct. 6, 2025

Related U.S. Application Data
(60) Provisional application No. 63/708,195, filed on Oct. 16, 2024.

(51) Int. Cl. C10L 3/00; C10L 3/10

(52) U.S. Cl. CPC C10L 3/003; C10L 3/10; C10L 2230/08

(57) ABSTRACT

The present disclosure generally relates to antifoulant compositions and methods of preventing or inhibiting fouling. The disclosure relates to antifoulant compositions comprising one or more solvents, one or more antipolymerants, and one or more dispersants. The compositions may include other optional components, such as an antioxidant, an alcohol, and/or water. Methods of using the compositions in natural gas dehydrogenation processes to prevent or minimize fouling are also disclosed.

### TECHNICAL FIELD

The present disclosure generally relates to antifoulant compositions and methods of preventing or inhibiting fouling. More particularly, the disclosure relates to antifoulant compositions comprising one or more solvents, one or more antipolymerants, and one or more dispersants and methods of using the compositions in natural gas dehydrogenation processes to prevent or minimize fouling.

### BACKGROUND

Cracking abundant natural gas feedstocks and purifying the resultant value-added products (e.g., ethylene, propylene, etc.) involves a wide temperature range. For example, process temperatures can span from about −60° C. to as high as about 600° C. Heavy hydrocarbons are formed as byproducts during the cracking process. The heavy byproducts include hot and flowable resins, tars and coke-like solids. On cooling, the previously flowable solid byproducts are deposited on the surfaces of the process equipment, such as transfer lines, compressors, compressor interstage coolers, and knockout drums, thereby causing severe fouling.

Reactive vinylic species, such as butadiene, are also byproducts. In the compressor units, these reactive monomers condense into films. The high pressure and high surface temperatures in the compressors cause the reactive monomers to polymerize into foulants. The compressed process gas is then cooled to cryogenic temperatures prior to fractional distillation of the byproducts.

Fractional distillation of vinylic species typically leads to the fouling of the fractionation reboilers due to undesirable polymerization of butadiene. Equipment fouling is replete throughout the value chain. Mitigating fouling in a process having such a wide range of operational temperatures presents many challenges. For example, current antifoulant compositions tend to freeze under the cryogenic temperatures.

Thus, to clean fouled equipment, operators are forced to shut down operations for the mechanical removal of the foulants. Shutdowns lead to lost production and lost revenue. Furthermore, the personnel responsible for the maintenance of the equipment are vulnerable to exposure to toxic chemicals. Reducing the frequency of unscheduled plant shutdowns and, also, ensuring personnel safety, gas processors require antifoulant technologies that reduce fouling so that the process efficiency is maintained, vis extending the operational time. The longer the operational period, or runlength, the lower the maintenance costs. Likewise, the risk of exposing the plant-maintenance personnel to chemicals is reduced.

### BRIEF SUMMARY

The present disclosure provides methods and compositions for inhibiting fouling.

In some embodiments, the disclosure provides a method of inhibiting fouling in a natural gas dehydrogenation process. The method comprises adding an antifoulant composition to a medium in the natural gas dehydrogenation process, wherein the antifoulant composition comprises a solvent, an antipolymerant, and a dispersant.

In some embodiments, an antifoulant composition of the present disclosure comprises a solvent, an antipolymerant, and a dispersant, wherein the solvent is selected from the group consisting of a carboxylic acid ester solvent, a cyclic ester, a phthalic acid ester, a lactone, a hydroxy carboxylate ester, an ester of a cyclohexane dicarboxylic acid, an ester of a cyclopentane dicarboxylic acid, a lactam, N-pyrolidone, a glycol, and any combination thereof.

The foregoing has outlined rather broadly the features and technical advantages of the present disclosure in order that the detailed description that follows may be better understood. Additional features and advantages of the disclosure will be described hereinafter that form the subject of the claims of this application.

### BRIEF DESCRIPTION OF THE SEVERAL VIEWS OF THE DRAWINGS

A detailed description of the invention is hereafter described with specific reference being made to the drawings in which:

FIG. 1 shows a schematic diagram of a propane dehydrogenation process.

### DETAILED DESCRIPTION

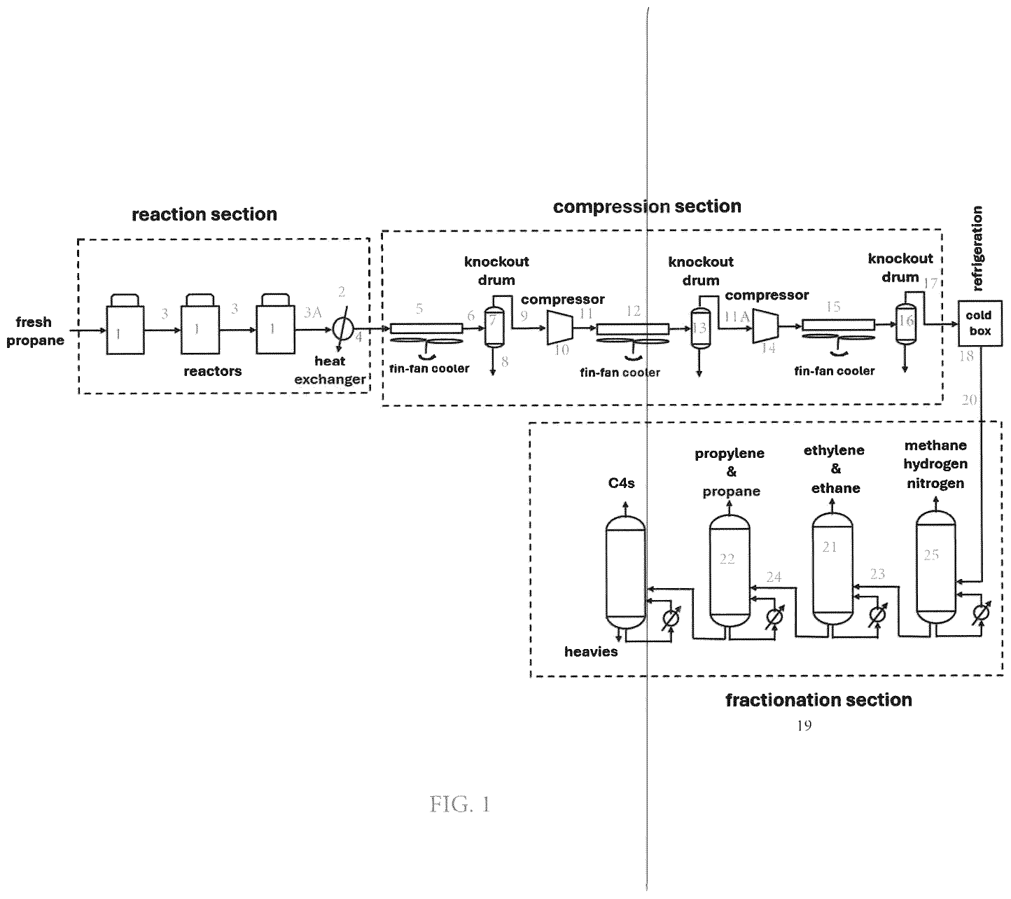

Various embodiments are described below and shown in FIG. 1. The relationship and functioning of the various elements of the embodiments will be better understood in light of the following detailed description. However, elements and embodiments are not strictly limited to those explicitly described below or shown in the drawing FIGURE.

Examples of methods and materials are described below, although methods and materials similar or equivalent to those described herein can be used in practice or testing of the present disclosure. All publications, patent applications, patents and other reference materials mentioned herein are incorporated by reference in their entirety. The materials, methods, and examples disclosed herein are illustrative only and not intended to be limiting.

Unless otherwise defined, all technical and scientific terms used herein have the same meaning as commonly understood by one of ordinary skill in the art. In case of conflict, the present document, including definitions, will control.

Unless otherwise indicated, an alkyl group as described herein alone or as part of another group is an optionally substituted linear or branched saturated monovalent hydrocarbon substituent containing from, for example, one to about sixty carbon atoms, such as one to about thirty carbon atoms, in the main chain. Examples of unsubstituted alkyl groups include methyl, ethyl, n-propyl, i-propyl, n-butyl, i-butyl, s-butyl, t-butyl, n-pentyl, i-pentyl, s-pentyl, t-pentyl, and the like.

The terms “aryl” or “ar” as used herein alone or as part of another group (e.g., arylene) denote optionally substituted homocyclic aromatic groups, such as monocyclic or bicyclic groups containing from about 6 to about 12 carbons in the ring portion, such as phenyl, biphenyl, naphthyl, substituted phenyl, substituted biphenyl or substituted naphthyl. The term “aryl” also includes heteroaryl functional groups. It is understood that the term “aryl” applies to cyclic substituents that are planar and comprise 4n+2 electrons, according to Huckel's Rule.

“Cycloalkyl” refers to a cyclic alkyl substituent containing from, for example, about 3 to about 8 carbon atoms, such as from about 4 to about 7 carbon atoms or about 4 to 6 carbon atoms. Examples of such substituents include cyclopropyl, cyclobutyl, cyclopentyl, cyclohexyl, cycloheptyl, cyclooctyl, and the like. The cyclic alkyl groups may be unsubstituted or further substituted with alkyl groups, such as methyl groups, ethyl groups, and the like.

“Heteroaryl” refers to a monocyclic or bicyclic 5- or 6-membered ring system, wherein the heteroaryl group is unsaturated and satisfies Huckel's rule. Non-limiting examples of heteroaryl groups include furanyl, thiophenyl, pyrrolyl, pyrazolyl, imidazolyl, 1,2,3-triazolyl, 1,2,4-triazolyl, isoxazolyl, oxazolyl, isothiazolyl, thiazolyl, 1,3,4-oxadiazol-2-yl, 1,2,4-oxadiazol-2-yl, 5-methyl-1,3,4-oxadiazole, 3-methyl-1,2,4-oxadiazole, pyridinyl, pyrimidinyl, pyrazinyl, triazinyl, benzofuranyl, benzothiophenyl, indolyl, quinolinyl, isoquinolinyl, benzimidazolyl, benzoxazolinyl, benzothiazolinyl, quinazolinyl, and the like.

Compounds of the present disclosure may be substituted with suitable substituents. The term “suitable substituent,” as used herein, is intended to mean a chemically acceptable functional group, preferably a moiety that does not negate the activity of the compounds. Such suitable substituents include, but are not limited to, halo groups, perfluoroalkyl groups, perfluoro-alkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkoxycarbonyl groups, alkylaminocarbonyl groups, dialkylamino carbonyl groups, arylcarbonyl groups, aryloxy-carbonyl groups, alkylsulfonyl groups, and arylsulfonyl groups. In some embodiments, suitable substituents may include halogen, an unsubstituted C₁-C₁₂ alkyl group, an unsubstituted C₄-C₆ aryl group, or an unsubstituted C₁-C₁₀ alkoxy group. Those skilled in the art will appreciate that many substituents can be substituted by additional substituents.

The term “substituted” as in “substituted alkyl,” means that in the group in question (e.g., the alkyl group), at least one hydrogen atom bound to a carbon atom is replaced with one or more substituent groups, such as hydroxy (—OH), alkylthio, phosphino, amido (—CON(R_{A})(R_{B}), wherein R_{A} and R_{B} are independently hydrogen, alkyl, or aryl), amino(—N(R_{A})(R_{B}), wherein R_{A} and R_{B} are independently hydrogen, alkyl, or aryl), halo (fluoro, chloro, bromo, or iodo), silyl, nitro (—NO₂), an ether (—OR_{A} wherein R_{A} is alkyl or aryl), an ester (—OC(O)R_{A} wherein R_{A} is alkyl or aryl), keto (—C(O)R_{A} wherein R_{A} is alkyl or aryl), heterocyclo, and the like.

When the term “substituted” introduces a list of possible substituted groups, it is intended that the term apply to every member of that group. That is, the phrase “optionally substituted alkyl or aryl” is to be interpreted as “optionally substituted alkyl or optionally substituted aryl.”

The terms “polymer,” “copolymer,” “polymerize,” “copolymerize,” and the like include not only polymers comprising two monomer residues and polymerization of two different monomers together, but also include (co)polymers comprising more than two monomer residues and polymerizing together more than two or more other monomers. For example, a polymer as disclosed herein includes a terpolymer, a tetrapolymer, polymers comprising more than four different monomers, as well as polymers comprising, consisting of, or consisting essentially of two different monomer residues. Additionally, a “polymer” as disclosed herein may also include a homopolymer, which is a polymer comprising a single type of monomer unit.

Unless specified differently, the polymers of the present disclosure may be linear, branched, crosslinked, structured, synthetic, semi-synthetic, natural, organic, inorganic, and/or functionally modified. A polymer of the present disclosure can be in the form of a solution, a dry powder, a liquid, or a dispersion, for example.

The term “natural gas” as used herein relates to a gas comprising methane, ethane, propane, butane, carbon dioxide, oxygen, nitrogen, or any combination thereof.

“Inhibiting fouling” as disclosed herein refers to, for example, reducing fouling, completely eliminating fouling or prohibiting fouling from occurring for some period of time, lowering a rate of fouling, etc.

With increasingly abundant natural gas becoming an economically advantageous feedstock for the production of high-value ethylene and propylene, petrochemical plants are experiencing high and frequent maintenance costs due to fouling. Apart from the targeted and desirable products, the dehydrogenation process leads to the formation of unwanted byproducts, including tars & polynuclear aromatics, coke-like solids, and reactive vinylic species, such as butadiene.

Some or all of the unwanted byproducts formed in the dehydrogenation process remain flowable as long as the temperature is elevated. However, a slight decrease in temperature leads to the formation of highly viscous tars and hard coke-like materials. The solids settle onto the surfaces of the process equipment, such as transfer lines, heat exchangers, compressors, compressor intercooler stages, and compressor knockout drums, and the overall process efficiency suffers due to the reduction of heat transfer and blocked transfer lines, for instance.

In some embodiments, the equipment is selected from the group consisting of a transfer line, a heat exchanger, a compressor, a knockout drum, a cooler, a reboiler, a reflux drum, a distillation column feed tray, a distillation column bottom tray, a fractionation column top section, and any combination thereof.

The presently disclosed antifoulant compositions inhibit, mitigate, eliminate, etc., fouling caused by such unwanted byproducts.

For example, with respect to FIG. 1, a schematic of a typical propane dehydrogenation process is shown, although the presently disclosed methods and compositions can be applied to any natural gas dehydrogenation process. The raw, natural gas feedstock is transported to one or more reactors (1) where it contacts a catalyst. As an illustrative example, if the feedstock comprises propane, the propane contacts the catalyst and is converted to propylene by the removal of two hydrogen atoms. If, for example, ethane is used as the feedstock, upon contacting the catalyst, it will be converted to ethylene.

After exiting the reactor, the converted feedstock (e.g., feedstock comprising ethylene, propylene, etc.) is transported to a heat exchanger (2). Temperatures are typically around 600° C. (slightly cooler than inside of the reactor) as the converted feedstock enters the heat exchanger (2). While FIG. 1 depicts an operation including one heat exchanger (2), multiple heat exchangers may be present and each one may be in communication with the reactor (1) via a transfer line.

The converted feedstock may contain unwanted byproducts, such as viscous tars and hard, coke-like materials. The solids can settle onto the surfaces of the process equipment, such as the reactor (1), the heat exchanger (2), and/or the transfer lines (3) between the reactor(s) and the heat exchanger (2). The transfer line downstream from the last reactor (1) leading to the heat exchanger (2) is indicated as transfer line (3A). While FIG. 1 depicts three reactors (3), the presently disclosed technology can be applied to dehydrogenation plants comprising any number of reactors, such as one, two, three, etc.

From the heat exchanger (2), the converted feedstock travels through transfer line (4), where temperatures may be around 145° C., to a cooler (5), such as a fin-fan cooler. This is the first point in the process where the converted feedstock is actively cooled. Upon cooling, foulants in the converted feedstock may solidify and deposit onto surfaces of the cooler (5).

After leaving the cooler (5), the converted feedstock travels through transfer line (6), where temperatures may be around 38° C., to knockout drum (7). In the knockout drum (7), unwanted liquids and solids in the converted feedstock may settle to the bottom (and exit the drum via transfer line (8)) while the gaseous converted feedstock may travel to and out the top through transfer line (9), where temperatures may be around 38° C. However, in certain circumstances, the gaseous converted feedstock exiting the drum (7) through transfer line (9) may still contain various amounts unwanted byproducts, such as droplets and/or solids, as a result of the fast-flowing gas. Such unwanted byproducts are capable of causing fouling in downstream transfer lines, compressors, etc.

Transfer line (9) leads the converted feedstock to a compressor (10) to reduce the volume of the process gas for easier handling and processing.

If left untreated, fouling is likely to occur in the compressors. Unlike conventional primary ethylene processes, propane dehydrogenation plants do not have units that quench the hot gas to cool it down and also knock down tars and solids prior to the transmission of the process stream to the compressor section. Thus, the tars and solids are entrained with the gas. Compressors are vulnerable to fouling from the byproducts emanating from the dehydrogenation reaction. Additionally, the high pressure leads to the condensation of some of the hydrocarbon byproducts, of which butadiene is an example. At high pressure, some of the heavy gaseous component condense to form films on the hot surfaces of the compressor equipment. At the elevated temperature, reactive vinylic species, such as butadiene, in the liquid films form unwanted polymers due to free radical polymerization. Thus, compressors are not only prone to fouling due to the tars and solids carried over from the reaction but fouling caused by the unwanted polymerization in the compressors as well.

The converted feedstock then exits the compressor through transfer line (11). Temperatures encountered in transfer line (11) may be around 135° C. since temperatures are elevated in the compressor. FIG. 1 depicts a second knockout drum (13), a second compressor (14), a second cooler (15), and corresponding transfer lines. Any number of knockout drums, compressors, and coolers may be present. After leaving knockout drum (16) through transfer line (17), the converted feedstock is transported to a refrigeration unit (18), where it is held until it is transported to the fractionation section (19) via transfer line (2) to undergo fractional distillation.

To separate the gases, the process stream is cooled to cryogenic conditions prior to separation by fractional distillation. During the distillation process, reboilers and bottom trays of the fractionation columns are prone to fouling through unwanted free-radical polymerization of reactive vinylic species in the process gas. Antifoulant compositions may be used to prevent unwanted polymerization. Specifically, the antifoulants may contact the cold process gas stream from the cryogenic unit. Unless they are stable at cryogenic temperatures associated with the process, the antifoulants will freeze. As a result of this low-temperature instability, the incumbent/prior art antifoulants tend to form solids. The solids block injection quills and transfer lines. Thus, the antifoulant is not delivered to the reboilers. To prevent the antifoulants from freezing, compositions that are stable at cryogenic conditions are required as ethylene and propylene producers increase the cracking of natural gas as feedstock.

The antifoulant compositions of the present disclosure comprise thermally and physically stable antifoulant compounds such that they may withstand temperatures up to around 650° C. and as low as about −60° C. (e.g., little or no evaporation at the elevated temperatures while having little or no freezing/solid formation at the lower temperatures). The antifoulant compositions of the present disclosure are capable of reducing or inhibiting fouling caused by different types of foulants (e.g., tars, polymers, solids, etc.)

For example, for abating fouling caused by tars and coke-like solids coming in the high-temperature effluent from the dehydrogenation reaction, thermally stable antifoulant compositions are provided. Thermal stability entails high boiling points as well as the maintenance of chemical integrity at the high temperatures associated with reaction effluent gas and compressor units.

The antifoulant compositions of the present disclosure comprise one or more solvents, one or more antipolymerants, and one or more dispersants. An antifoulant composition may optionally further comprise an antioxidant and/or an alcohol, such as amyl alcohol. To be effective, the solvents in the compositions have high solvency and high boiling points, such as boiling points from about 0° C. to about 650° C., about 50° C. to about 650° C., about 100° C. to about 650° C., about 150° C. to about 650° C., about 200° C. to about 650° C., about 250° C. to about 650° C., about 300° C. to about 650° C., about 350° C. to about 650° C., about 400° C. to about 650° C., about 450° C. to about 650° C., about 500° C. to about 650° C., about 550° C. to about 650° C., or about 600° C. to about 650° C.

The solvents are also stable (e.g., do not form solids) at lower temperatures, such as about 0° C. to about −60° C., −10° C. to about −60° C., −20° C. to about −60° C., −30° C. to about −60° C., −40° C. to about −60° C., or −50° C. to about −60° C.

The dispersants prevent the fouling of the process equipment caused by the precipitation of the solids from the reactor section. Stable free-radical species (e.g., antipolymerants) are included for the purpose of preventing the formation of unwanted polymers.

Illustrative, non-limiting examples of solvents include aromatic solvents, carboxylic acid ester solvents, cyclic esters, phthalic acid esters, lactones, hydroxy carboxylate esters, esters of cyclohexane dicarboxylic acids (1,2; 1,3; and 1,4), esters of cyclopentane dicarboxylic acids (1,2; 1,3; and 1,4), a lactam, N-pyrolidone, a glycol (e.g., ethylene glycol), or any combination thereof.

In some embodiments, the solvent comprises heavy aromatic naphtha, ethyl caprylate, octyl acetate, dimethyl phthalate, 2-ethlyhexyl acetate, or any combination thereof.

In certain embodiments, the solvent comprises a carboxylic acid, such as a carboxylic acid comprising the formula

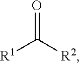

wherein R¹ is a C₁-C₁₈ alkyl group, a C₁-C₁₈ aryl alkyl group, or a C₁-C₁₈ alkyl aryl group; and R² is a C₁-C₁₈ alkyl group, a C₁-C₁₈ aryl alkyl group, or a C₁-C₁₈ alkyl aryl group.

In some embodiments, the solvent comprises ethyl caprylate, octyl acetate, 2-ethlyhexyl acetate, or a combination thereof.

In certain embodiments, the solvent comprises a dialkyl phthalate (e.g., dimethyl phthalate) having the following formula:

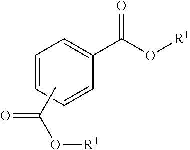

wherein each R¹ is independently selected from a C₁-C₁₈ alkyl group, a C₁-C₁₈ aryl alkyl group, or a C₁-C₁₈ alkyl aryl group. For example, each R¹ may be independently selected from a C₁-C₁₅ alkyl group, a C₁-C₁₂ alkyl group, C₁-C₉ alkyl group, a C₁-C₆ alkyl group, a C₁-C₃ alkyl group, a C₁-C₁₅ aryl alkyl group, a C₁-C₁₂ aryl alkyl group, a C₁-C₉ aryl alkyl group, a C₁-C₆ aryl alkyl group, a C₁-C₃ aryl alkyl group, a C₁-C₁₅ alkyl aryl group, a C₁-C₁₂ alkyl aryl group, a C₁-C₉ alkyl aryl group, a C₁-C₆ alkyl aryl group, or a C₁-C₃ alkyl aryl group.

In some embodiments, the solvent comprises a dialkyl 1,2-cyclohexyldicarboxylate having the formula:

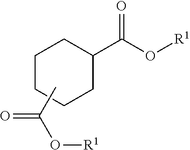

wherein each R¹ is independently selected from a C₁-C₁₈ alkyl group. For example, each R¹ may be independently selected from a C₁-C₁₅ alkyl group, a C₁-C₁₂ alkyl group, C₁-C₉ alkyl group, a C₁-C₆ alkyl group, or a C₁-C₃ alkyl group.

In some embodiments, a composition of the present disclosure comprises a solvent selected from ethyl caprylate, octyl acetate, 2-ethlyhexyl acetate. Such a composition can be advantageously added at any location depicted in FIG. 1, although any composition disclosed herein may be added at any location depicted in FIG. 1.

In some embodiments, a composition of the present disclosure comprises a solvent selected from heavy aromatic naphtha, toluene, xylene, alkyl benzenes, butyl glycol, diethylene glycol monobutyl ether, or a combination thereof. In certain embodiments, such a composition may be added, for example, at any location downstream of the final reactor (1) (e.g., at transfer line 3A) and upstream of knockout drum (16) (e.g., at transfer line 11A).

In some embodiments, the solvent excludes methanol.

A composition of the present disclosure may comprise any amount of the solvent. For example, a composition may comprise from about 1 wt. % to about 99 wt. % of the solvent, such as from about 10 wt. % to about 99 wt. %, about 20 wt. % to about 99 wt. %, about 30 wt. % to about 99 wt. %, about 40 wt. % to about 99 wt. %, about 50 wt. % to about 99 wt. %, about 60 wt. % to about 99 wt. %, about 70 wt. % to about 99 wt. %, about 80 wt. % to about 99 wt. %, or about 90 wt. % to about 99 wt. %.

Illustrative, non-limiting examples of an antipolymerant include 2,2,6,6-tetramethylpiperidinyl-1-oxyl (TEMPO), 4-hydroxy-2,2,6,6-tetramethylpiperidinyl-1-oxyl (HTMPO), 4-oxo-2,2,6,6-tetramethylpiperidinyl-1-oxyl (OTEMPO), di-tert-butyl nitroxyl, 1-oxyl-2,2,6,6-tetramethyl-4-n-propoxypiperidine, 1-oxyl-2,2,6,6-tetramethyl-4-n-butoxypiperidine, 1-oxyl-2,2,6,6-tetramethyl-4-t-butoxypiperidine, 1-oxyl-2,2,6,6-tetramethyl-4-s-butoxypiperidine, 1-oxyl-2,2,6,6-tetramethyl-4-(2-methoxyethoxy)piperidine, 1-oxyl-2,2,6,6-tetramethyl-4-(2-methoxyethoxyacetoxy)piperidine, 1-oxyl-2,2,6,6-tetramethylpiperidin-4-yl stearate, 1-oxyl-2,2,6,6-tetramethylpiperidin-4-yl acetate, 1-oxyl-2,2,6,6-tetramethylpiperidin-4-yl butyrate, 1-oxyl-2,2,6,6-tetramethylpiperidin-4-yl 2-ethylhexanoate, 1-oxyl-2,2,6,6-tetramethylpiperidin-4-yl octanoate, 1-oxyl-2,2,6,6-tetramethylpiperidin-4-yl laurate, 1-oxyl-2,2,6,6-tetramethylpiperidin-4-yl benzoate, 1-oxyl-2,2,6,6-tetramethylpiperidin-4-yl 4-tert-butylbenzoate, 1-oxyl-2,2,6,6-tetramethyl-4-allyloxy-piperidine, 1-oxyl-2,2,6,6-tetramethyl-4-acetamidopiperidine, 1-oxyl-2,2,6,6-tetramethyl-4-(N-butylformamido)piperidine, N-(1-oxyl-2,2,6,6-tetramethylpiperidin-4-yl)-caprolactam, N-(1-oxyl-2,2,6,6-tetramethylpiperidin-4-yl)-dodecylsuccinimide, 1-oxyl-2,2,6,6-tetramethyl-4-(2,3-dihydroxypropoxy)piperidine, 1-oxyl-2,2,6,6-tetramethyl-4-(2-hydroxyl-4-oxapentoxy)piperidine, and any combination thereof.

Additional examples include bis(1-oxyl-2,2,6,6-tetramethylpiperidin-4-yl) succinate, bis(1-oxyl-2,2,6,6-tetramethylpiperidin-4-yl) adipate, bis(1-oxyl-2,2,6,6-tetramethylpiperidin-4-yl) sebacate, bis(1-oxyl-2,2,6,6-tetramethylpiperidin-4-yl) n-butylmalonate, bis(1-oxyl-2,2,6,6-tetramethylpiperidin-4-yl) phthalate, bis(1-oxyl-2,2,6,6-tetramethylpiperidin-4-yl) isophthalate, bis(1-oxyl-2,2,6,6-tetramethylpiperidin-4-yl) terephthalate, bis(1-oxyl-2,2,6,6-tetramethylpiperidin-4-yl) hexahydroterephthalate, N,N′-bis(1-oxyl-2,2,6,6-tetramethylpiperidin-4-yl) adipamide, 2,4,6-tris-[N-butyl-N-(1-oxyl-2,266-tetramethylpiperidin-4-yl)]-s-triazine, 2,4,6-tris-[N-(1-oxyl-2,2,6,6-tetramethylpiperidin-4-yl)]-s-triazine, 4,4′-ethylenebis(1-oxyl-2,2,6,6-tetramethylpiperazin-3-one), and any combination thereof.

An antipolymerant may comprise a quinone methide, such as a quinone methide having the following structure:

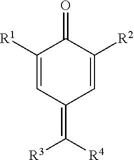

wherein R¹ and R² are independently selected from a C₄-C₁₈ alkyl group, a C₅-C₁₂ cycloalkyl group, a phenyl group, and a C₇-C₁₅ cycloalkyl group, wherein R³ and R⁴ are independently selected from —H, a C₁-C₁₈ alkyl group, a phenyl group, a substituted phenyl group, a C₅-C₁₂ cycloalkyl group, —CN, —COOH, —C≡CR⁵, —C≡CR⁵, —COOR⁵, —COR⁵, —OCOR⁵, —CONR⁵, wherein R⁵ is selected from H, a C₁-C₁₈ alkyl group, a C₅-C₁₂ cycloalkyl group, a phenyl group, a C₇-C₁₅ cycloalkyl group, and a substituted phenyl group.

In some embodiments, the antipolymerant is selected from the group consisting of a nitroxide-containing compound comprising a piperidinol group of the following formula:

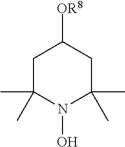

wherein R⁸ is selected from H, a C₁-C₂₂ alkyl group, a C₁-C₂₂ aryl group, a C₁-C₂₂ aryl alkyl group, or a C₁-C₂₂ alkyl aryl group. For example, R⁸ may be selected from a C₁-C₁₈ alkyl group, a C₁-C₁₅ alkyl group, C₁-C₁₁ alkyl group, a C₁-C₇ alkyl group, a C₁-C₃ alkyl group, a C₁-C₁₈ aryl group, a C₁-C₁₅ aryl group, C₁-C₁₁ aryl group, a C₁-C₇ aryl group, a C₁-C₃ aryl group, a C₁-C₁₈ aryl alkyl group, a C₁-C₁₅ aryl alkyl group, a C₁-C₁₁ aryl alkyl group, a C₁-C₇ aryl alkyl group, a C₁-C₃ aryl alkyl group, a C₁-C₁₈ alkyl aryl group, a C₁-C₁₅ alkyl aryl group, a C₁-C₁₁ alkyl aryl group, a C₁-C₇ alkyl aryl group, or a C₁-C₃ alkyl aryl group.

In some embodiments, the antipolymerant is selected from the group consisting of a nitroxide-containing compound comprising a piperidinoxy group of the following formula:

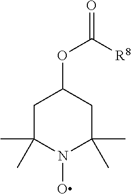

wherein R⁸ is selected from H, a C₁-C₂₂ alkyl group, a C₁-C₂₂ aryl group, a C₁-C₂₂ aryl alkyl group, or a C₁-C₂₂ alkyl aryl group. For example, R⁸ may be selected from a C₁-C₁₈ alkyl group, a C₁-C₁ alkyl group, C₁-C₁₁ alkyl group, a C₁-C₇ alkyl group, a C₁-C₃ alkyl group, a C₁-C₁₈ aryl group, a C₁-C₁ aryl group, C₁-C₁₁ aryl group, a C₁-C₇ aryl group, a C₁-C₃ aryl group, a C₁-C₁₈ aryl alkyl group, a C₁-C₁ aryl alkyl group, a C₁-C₁₁ aryl alkyl group, a C₁-C₇ aryl alkyl group, a C₁-C₃ aryl alkyl group, a C₁-C₁₈ alkyl aryl group, a C₁-C₁ alkyl aryl group, a C₁-C₁₁ alkyl aryl group, a C₁-C₇ alkyl aryl group, or a C₁-C₃ alkyl aryl group.

In some embodiments, the antipolymerant is selected from the group consisting of a nitroxide-containing compound comprising a piperidinol group of the following formula:

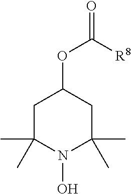

wherein R⁸ is selected from H, a C₁-C₂₂ alkyl group, a C₁-C₂₂ aryl group, a C₁-C₂₂ aryl alkyl group, or a C₁-C₂₂ alkyl aryl group. For example, R⁸ may be selected from a C₁-C₁₈ alkyl group, a C₁-C₁₅ alkyl group, C₁-C₁₁ alkyl group, a C₁-C₇ alkyl group, a C₁-C₃ alkyl group, a C₁-C₁₈ aryl group, a C₁-C₁₅ aryl group, C₁-C₁₁ aryl group, a C₁-C₇ aryl group, a C₁-C₃ aryl group, a C₁-C₁₈ aryl alkyl group, a C₁-C₁₅ aryl alkyl group, a C₁-C₁₁ aryl alkyl group, a C₁-C₇ aryl alkyl group, a C₁-C₃ aryl alkyl group, a C₁-C₁₈ alkyl aryl group, a C₁-C₁₅ alkyl aryl group, a C₁-C₁₁ alkyl aryl group, a C₁-C₇ alkyl aryl group, or a C₁-C₃ alkyl aryl group.

In some embodiments, an antipolymerant comprises 4-hydroxy-2,2,6,6-tetramethylpiperidyl-1-oxyl, 1,4-dihydroxy-2,2,6,6-tetramethylpiperidyl-1-oxyl, or any combination thereof.

A composition of the present disclosure may comprise any amount of the antipolymerant. For example, a composition may comprise from about 1 wt. % to about 99 wt. % of the antipolymerant, such as from about 1 wt. % to about 90 wt. %, from about 1 wt. % to about 80 wt. %, about 1 wt. % to about 70 wt. %, about 1 wt. % to about 60 wt. %, about 1 wt. % to about 50 wt. %, about 1 wt. % to about 40 wt. %, about 1 wt. % to about 30 wt. %, about 1 wt. % to about 20 wt. %, about 1 wt. % to about 10 wt. %, about 1 wt. % to about 8 wt. %, about 1 wt. % to about 6 wt. %, about 1 wt. % to about 4 wt. %, or about 1 wt. % to about 2 wt. %.

Illustrative, non-limiting examples of dispersants include an imidazoline, a fatty acid hydroxyalkylamine ester (such as trimethylamine (TEA)—tall oil fatty acid (TOFA)/soy fatty acid (SOFA)/castor oil fatty acid (COFA)), a polymer comprising an α-olefin, a polymer comprising maleic anhydride, a polymer comprising an α-olefin and a maleic anhydride in which one or more (or all) of the maleic anhydride residues are reacted with a primary amine, a styrene sulfonate polymer, a lignosulfonate metal salt, a naphthalene sulfonate formaldehyde condensate polymer, an acrylate polymer, or any combination thereof.

In some embodiments, the imidazoline comprises the following structure:

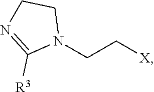

wherein the R³ group is a C₁-C₁₈ alkyl group, such as a C₁-C₁₅ alkyl group, a C₁-C₁₂ alkyl group, a C₁-C₈ alkyl group, or a C₁-C₄ alkyl group, X is NHR¹, wherein R¹ is H or a C₁-C₁₈ alkyl group, such as a C₁-C₁₅ alkyl group, a C₁-C₁₁ alkyl group, a C₁-C₈ alkyl group, or a C₁-C₄ alkyl group, or X is OR¹, wherein R¹ is H or a C₁-C₁₈ alkyl group, a C₁-C₁₄ alkyl group, a C₁-C₁₁ alkyl group, a C₁-C₇ alkyl group, or a C₁-C₃ alkyl group.

In some embodiments, the copolymer of an α-olefin and maleic anhydride comprises the following formula:

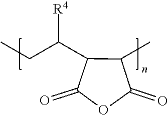

wherein R⁴ is H or a C₁-C₁₈ alkyl group, such as a C₁-C₁ alkyl group, a C₁-C₁₂ alkyl group, a C₁-C₈alkyl group, or a C₁-C₄ alkyl group; and n is an integer of 10 or more, such as an integer selected from 10 to 25, 10 to 50, 10 to 75, 10 to 100, 10 to 200, 10 to 300, or 10 to more than 300.

In some embodiments, the amino adduct of the copolymer of an α-olefin and maleic anhydride comprises the following formula:

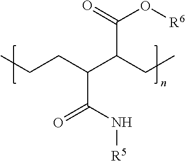

wherein R⁵ is a C₁-C₁₈ alkyl group, such as a C₁-C₁₁ alkyl group or a C₁-C₅ alkyl group, a C₆-C₁₈ aryl alkyl group, such as a C₆-C₁₄ aryl alkyl group or a C₆-C₁₀ aryl alkyl group, a C₁-C₁₈ alkyl aryl group, such as a C₁-C₁₁ alkyl aryl group or a C₁-C₅ alkyl aryl group, or an amino C₆-C₁₈ aryl alkyl group, such as an amino C₆-C₁₂ aryl alkyl group or an amino C₁₂-C₁₈ aryl alkyl group; R⁶ is H or a C₁-C₁₈ alkyl group, such as a C₁-C₁₅ alkyl group, a C₁-C₁₂ alkyl group, a C₁-C₈alkyl group, or a C₁-C₄ alkyl group; and n is an integer of 10 or more, such as an integer selected from 10 to 25, 10 to 50, 10 to 75, 10 to 100, 10 to 200, 10 to 300, or 10 to an integer greater than 300.

In some embodiments, the dispersant comprises a hydroxy adduct of a copolymer of an α-olefin and maleic anhydride having the following structure:

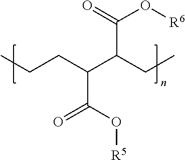

wherein R⁵ is a C₁-C₁₈ alkyl group, such as a C₁₀-C₁₈ alkyl group or a C₁-C₉ alkyl group, a C₆-C₁₈ aryl alkyl group, such as a C₁₁-C₁₈ aryl alkyl group or a C₆-C₁₀ aryl alkyl group, a C₁-C₁₈ alkyl aryl group, such as a C₁-C₈alkyl aryl group or a C₁₁-C₁₈ alkyl aryl group, or a C₆-C₁₈ aryl alkyl group, such as a C₆-C₁₁ aryl alkyl group or a C₁₂-C₁₈ aryl alkyl group; R⁶ is H or a C₁-C₁₈ alkyl group, such as a C₁-C₁₄ alkyl group, a C₁-C₁₀ alkyl group, or a C₁-C₅ alkyl group; and n is an integer of 10 or more, such as an integer selected from 10 to 25, 10 to 50, 10 to 75, 10 to 100, 10 to 200, 10 to 300, or 10 to an integer greater than 300.

In some embodiments, the dispersant comprises a styrene sulfonate polymer having the following structure:

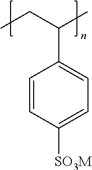

wherein M is H, an alkali metal, or an earth alkali metal; and n is an integer of 10 or more, such as an integer selected from 10 to 25, 10 to 50, 10 to 75, 10 to 100, 10 to 200, 10 to 300, or 10 to an integer greater than 300.

In some embodiments, the dispersant comprises a naphthalene sulfonate formaldehyde condensate polymer having the following structure:

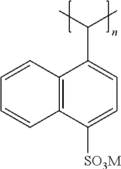

wherein M is H, an alkali metal, or an earth alkali metal; and n is an integer of 10 or more, such as an integer selected from 10 to 25, 10 to 50, 10 to 75, 10 to 100, 10 to 200, 10 to 300, or 10 to an integer greater than 300.

In some embodiments, the dispersant comprises a polyethoxylated p-alkylphenonol carbonyl condensate polymer having the following structure:

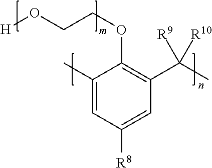

wherein R⁸ is a C₉-C₁₈ alkyl group, such as a C₉-C₁₂ alkyl group or a C₁₃-C₁₈ alkyl group, a C₉-C₁₈ aryl alkyl group, such as a C₉-C₁₃ aryl alkyl group or a C₁₄-C₁₈ aryl alkyl group, or a C₉-C₁₈ alkyl aryl group, such as a C₁₃-C₁₈ alkyl aryl group or a C₉-C₁₄ alkyl aryl group; R⁹ is H, a C₁-C₁₈ alkyl group, such as a C₁₂-C₁₈ alkyl group or a C₁-C₈ alkyl group, a C₁-C₁₈ aryl alkyl group, such as a C₁₂-C₁₈ aryl alkyl group or a C₁-C₈ aryl alkyl group, or a C₁-C₁₈ alkyl aryl group, such as a C₁-C₈ alkyl aryl group or a C₁₂-C₁₈ alkyl aryl group; R¹⁰ is H, a C₁-C₁₈ alkyl group, such as a C₁-C₈ alkyl group or a C₁₂-C₁₈ alkyl group, a C₁-C₁₈ aryl alkyl group, such as a C₁-C₈ aryl alkyl group or a C₁₂-C₁₈ aryl alkyl group, or a C₁-C₁₈ alkyl aryl group, such as a C₁₂-C₁₈ alkyl aryl group or a C₁-C₈ alkyl aryl group; m is an integer from 0 to 20, such as 1 to 15, 1 to 10, 1 to 5, 5 to 20, 10 to 20, or 15 to 20; and n is an integer from 4 to 75, such as 4 to 65, 4 to 55, 4 to 45, 4 to 35, 4 to 25, 4 to 15, 10 to 75, 20 to 75, 30 to 75, 40 to 75, 50 to 75, or 60 to 75.

A composition of the present disclosure may comprise any amount of the dispersant. For example, a composition may comprise from about 1 wt. % to about 99 wt. % of the dispersant, such as from about 1 wt. % to about 90 wt. %, from about 1 wt. % to about 80 wt. %, about 1 wt. % to about 70 wt. %, about 1 wt. % to about 60 wt. %, about 1 wt. % to about 50 wt. %, about 1 wt. % to about 40 wt. %, about 1 wt. % to about 30 wt. %, about 1 wt. % to about 20 wt. %, about 1 wt. % to about 10 wt. %, about 1 wt. % to about 8 wt. %, about 1 wt. % to about 6 wt. %, about 1 wt. % to about 4 wt. %, or about 1 wt. % to about 2 wt. %.

Illustrative, non-limiting examples of antioxidants include a phenylenediamine (e.g., ortho-phenylenediamine, meta-phenylenediamine, or para-phenylenediamine), a hydroquinone, a hydroxylamine, or any combination thereof.

Hydroquinones include, for example, Formula 1:

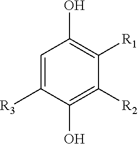

wherein R₁, R₂, and R₃ are independently hydrogen, alkyl, aryl, heterocyclo, or R₁ and R₂ together with the carbons they are attached to form a 5- or 6-membered fused cyclo, aryl, heterocyclo, or heteroaryl ring; wherein at least two of R₁, R₂, and R₃ are other than hydrogen.

A composition of the present disclosure may comprise any amount of the antioxidant. For example, a composition may comprise from about 0 wt. % to about 99 wt. % of the antioxidant, such as from about 1 wt. % to about 90 wt. %, from about 1 wt. % to about 80 wt. %, about 1 wt. % to about 70 wt. %, about 1 wt. % to about 60 wt. %, about 1 wt. % to about 50 wt. %, about 1 wt. % to about 40 wt. %, about 1 wt. % to about 30 wt. %, about 1 wt. % to about 20 wt. %, about 1 wt. % to about 10 wt. %, about 1 wt. % to about 8 wt. %, about 1 wt. % to about 6 wt. %, about 1 wt. % to about 4 wt. %, or about 1 wt. % to about 2 wt. %.

As mentioned earlier, a composition of the present disclosure may also comprise an alcohol. Illustrative, non-limiting examples of alcohols include amyl alcohol, n-propanol, isopropanol, n-butanol, sec-butanol, tert-butanol, or any combination thereof.

A composition of the present disclosure may comprise any amount of the alcohol. For example, a composition may comprise from about 0 wt. % to about 99 wt. % of the alcohol, such as from about 1 wt. % to about 90 wt. %, from about 1 wt. % to about 80 wt. %, about 1 wt. % to about 70 wt. %, about 1 wt. % to about 60 wt. %, about 1 wt. % to about 50 wt. %, about 1 wt. % to about 40 wt. %, about 1 wt. % to about 30 wt. %, about 1 wt. % to about 20 wt. %, about 1 wt. % to about 10 wt. %, about 20 wt. % to about 60 wt. %, about 30 wt. % to about 50 wt. %, or about 35 wt. % to about 45 wt. %.

A composition of the present disclosure may also comprise water. The amount of water in the composition is not particularly limited. In some embodiments, a composition of the present disclosure may comprise from about 0 wt. % to about 99 wt. % of water, such as from about 0.1 wt. % to about 50 wt. %, from about 0.1 wt. % to about 40 wt. %, about 0.1 wt. % to about 30 wt. %, about 0.1 wt. % to about 20 wt. %, about 0.1 wt. % to about 10 wt. %, about 0.1 wt. % to about 5 wt. %, about 0.1 wt. % to about 3 wt. %, about 0.1 wt. % to about 1 wt. %, about 1 wt. % to about 2 wt. %, about 1 wt. % to about 4 wt. %, about 1 wt. % to about 6 wt. %, or about 1 wt. % to about 10 wt. %.

In certain embodiments, an antifoulant composition of the present disclosure comprises ethyl caprylate, 4-hydroxy-2,2,6,6-tetramethylpiperidyl-1-oxyl, 1,4-dihydroxy-2,2,6,6-tetramethylpiperidyl-1-oxyl, water, and an alcohol, such as amyl alcohol.

In certain embodiments, an antifoulant composition of the present disclosure comprises octyl acetate, 4-hydroxy-2,2,6,6-tetramethylpiperidyl-1-oxyl, 1,4-dihydroxy-2,2,6,6-tetramethylpiperidyl-1-oxyl, water, and an alcohol, such as amyl alcohol.

In certain embodiments, an antifoulant composition of the present disclosure comprises dimethyl phthalate, 4-hydroxy-2,2,6,6-tetramethylpiperidyl-1-oxyl, 1,4-dihydroxy-2,2,6,6-tetramethylpiperidyl-1-oxyl, water, and an alcohol, such as amyl alcohol.

In certain embodiments, an antifoulant composition of the present disclosure comprises 2-ethylhexyl acetate, 4-hydroxy-2,2,6,6-tetramethylpiperidyl-1-oxyl, 1,4-dihydroxy-2,2,6,6-tetramethylpiperidyl-1-oxyl, water, and an alcohol, such as amyl alcohol.

The presently disclosed antifoulant compositions may be added at any location shown in FIG. 1. As illustrative, non-limiting examples, a composition of the present disclosure may be added at any transfer line, such as transfer line (3A), transfer line (4), transfer line (9), transfer line (11), transfer line (11A), transfer line (20), or any combination thereof.

In some embodiments, an antifoulant composition is added downstream from the last reactor (1) and upstream of the fractionation section (19) or a device located in the fractionation section, such as ethylene & ethane tank (21), propylene & propane tank (22), and/or any transfer line in the fractionation section (19).

In some embodiments, an antifoulant composition is added downstream from the last reactor (1) and upstream of knockout drum (16).

In certain embodiments, an antifoulant composition is added downstream from the last reactor (1) and upstream of compressor (14) or compressor (10).

In certain embodiments, an antifoulant composition is added downstream from a final knockout drum (16) or downstream from a refrigeration unit (18) and upstream of the fractionation section (19) or a device located in the fractionation section, such as ethylene & ethane tank (21), propylene & propane tank (22), and/or any transfer line in the fractionation section (19).

The fractionation section includes temperatures down to as low as about −60° C. During fractionation, unwanted polymers may be formed, which can cause fouling if not treated. Accordingly, there may be a need to add the presently disclosed antifoulant compositions to the fractionation section (19), such as to transfer line (20) and/or to any transfer line in the fractionation section (19), such as transfer line (23) between the methane/hydrogen/nitrogen tank (25) and the ethylene & ethane tank (21) or transfer line (24) between the ethylene & ethane tank (21) and the propylene & propane tank (22).

The antifoulant compositions disclosed herein may be added to, for example, a feedstock and/or a converted feedstock in an amount ranging from about 1 ppm to about 10,000 ppm, such as about 1 ppm to about 5,000 ppm, about 1 ppm to about 2,500 ppm, about 1 ppm to about 2,000 ppm, about 1 ppm to about 1,000 ppm, about 1 ppm to about 750 ppm, about 1 ppm to about 500 ppm, about 1 ppm to about 250 ppm, about 1 ppm to about 100 ppm, about 25 ppm to about 5,000 ppm, about 25 ppm to about 2,500 ppm, about 25 ppm to about 1,500 ppm, about 25 ppm to about 1,000 ppm, or about 25 ppm to about 500 ppm.

The compositions and/or compounds disclosed herein may be added to a medium (e.g., feedstock, converted feedstock, etc.) using a variety of different application methods known in the art. In some embodiments, the compositions and/or compounds may be added continuously or intermittently to the medium, either automatically or manually, by using, for example, chemical injection pumps. The addition may involve dripping, pouring, spraying, pumping, injecting, or otherwise adding the composition and/or compound to the medium.

The foregoing may be better understood by reference to the following examples, which are intended for illustrative purposes and are not intended to limit the scope of the disclosure or its application in any way.

### Examples

Five formulations were prepared for testing. In Formulation 1, 150 g of heavy aromatic naphtha, 5 g of 4-hydroxy-2,2,6,6-tetramethylpiperidyl-1-oxyl, 3 g of 1,4-dihydroxy-2,2,6,6-tetramethylpiperidyl-1-oxyl, 0.2 g of water and 41.8 g of amyl alcohol were added to a blender. The mixture was agitated until all of the solids went into solution. Formulation 2 was the same as Formulation 1 except ethyl caprylate was used instead of heavy aromatic naphtha. Formulation 3 was the same as Formulation 1 except octyl acetate was used instead of heavy aromatic naphtha. Formulation 4 was the same as Formulation 1 except dimethyl phthalate was used instead of heavy aromatic naphtha. Formulation 5 was the same as Formulation 1 except 2-ethylhexyl acetate was used instead of heavy aromatic naphtha. The formulations are shown in Table 1.

TABLE 1

| Components (g) | Form. 1 | Form. 2 | Form. 3 | Form. 4 | Form. 5 |
|---|---|---|---|---|---|
| Heavy aromatic naphtha | 150 | | | | |
| Ethyl Caprylate | | 150 | | | |
| Octyl Acetate | | | 150 | | |
| Dimethyl Phthalate | | | | 150 | |
| 2-EthlyHexyl Acetate | | | | | 150 |
| 4-Hydroxy-2,2,6,6- | 5 | 5 | 5 | 5 | 5 |
| Tetramethylpiperidyl-1- | | | | | |
| oxyl | | | | | |
| 1,4-Dihydroxy-2,2,6,6- | 3 | 3 | 3 | 3 | 3 |
| Tetramethylpiperidyl-1- | | | | | |
| oxyl | | | | | |
| Water | 0.2 | 0.2 | 0.2 | 0.2 | 0.2 |
| Amyl Alcohol | 41.8 | 41.8 | 41.8 | 41.8 | 41.8 |

The formulations listed in Table 1 were tested for low-temperature stability by incubating the sample at about −55° C. Owing to the undesirable consequences of using methanol to stabilize the antifoulant composition, heavy aromatic naphtha was replaced with ethyl caprylate, octyl acetate, dimethyl phthalate, and 2-ethlyhexyl acetate. Using sustainable organic solvents to replace heavy aromatic naphtha was found to impart surprising low-temperature stability to the antipolymerant formulation.

Formulation 1, Formulation 3, Formulation 4, and Formulation 5 were subsequently incubated at a temperature of about −55° C. Formulations 2 and 5 displayed remarkable physical stability at about −55° C. By comparison the incumbent product, Formulation 1, performed quite poorly. Also, freezing occurred in Formulation 4.

Next, dispersancy tests were conducted where a deposit was collected from the knock-out drum immediately upstream of a first stage gas compressor. The tar deposit had a paste-like texture. Into each of three 15-mL centrifuge tubes, 10 mL of heptane were added. One of the tubes was treated with dispersant Formulation 7 comprising about 15 wt. % of an α-olefin maleic anhydride polymer in heavy aromatic naphtha to give a dose of about 2,000 ppm. Similarly, the other tube was dosed with about 2,000 ppm of Formulation 8 comprising about 40 wt. % of a nonylphenol-formaldehyde polymer in heavy aromatic naphtha. The two tubes were shaken to homogenize the mixtures. The untreated tube was the blank since it contained no dispersant. To render it flowable, the tar-like deposit was incubated in a water bath at about 70° C. for about 60 minutes. Using a pipette, 500 μL of the molten foulant were added into each the three centrifuge tubes followed by shaking for about 30 seconds. The tubes were then placed in a test tube holder and the rate of the settling of the solids captured by taking pictures as a function of time.

According to the results, the effectiveness of the inventive formulations was demonstrated by a low rate of sedimentation of the dispersed foulant. In the untreated test, the dispersed solids quickly settled to the bottom of the test tube. Formulations 7 and 8 outperformed the blank, with Formulation 7 being the top performer. The treatment using Formulation 7 resulted in a sustained dispersion of the solids for longer than 30 minutes.

Next, washing efficacy tests were conducted. The effectiveness of the inventive compositions as washing compositions was tested using the following method. A coke-like deposit was taken from the reactor effluent line of a propane dehydrogenation plant and used to test the efficacy of the inventive compositions.

500-mg pieces of the hard deposit were placed into each of three scintillation vials. About 10 mL of Washing Composition 1 (comprising toluene) were added into a first vial, about 10 mL of Washing Composition 2 (comprising heavy aromatic naphtha) were added into a second vial, and about 10 mL of Washing Composition 3 (comprising dimethylphthalate) were added to a third vial. Without agitating the vials, the deposit pieces started to dissolve spontaneously. On agitation, the solids were almost completely dissolved.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a dispersant” is intended to include “at least one dispersant” or “one or more dispersants.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25° C. with neat (not diluted) polymers.

As used herein, the term “about” refers to the cited value being within the errors arising from the standard deviation found in their respective testing measurements, and if those errors cannot be determined, then “about” may refer to, for example, within 5%, 4%, 3%, 2%, or 1% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A method of inhibiting fouling in a natural gas dehydrogenation process, comprising:
adding an antifoulant composition to a medium in the natural gas dehydrogenation process, wherein the antifoulant composition comprises a solvent, an antipolymerant, and a dispersant.

2. The method of claim 1, further comprising adding the antifoulant composition to a location selected from the group consisting of a transfer line, downstream of a reactor, upstream of a knockout drum, upstream of a compressor, upstream of a fractionation section, downstream of a knockout drum, downstream of a refrigeration unit, upstream of an ethylene tank, upstream of a propylene tank, to a fractionation section, or any combination thereof.

3. The method of claim 1, wherein the natural gas dehydrogenation process is a propane dehydrogenation process.

4. The method of claim 1, wherein the solvent comprises a boiling point of about 650° C. or less and/or a freezing point less than about −60° C.

5. The method of claim 1, wherein the solvent is selected from the group consisting of an aromatic solvent, a carboxylic acid ester solvent, a cyclic ester, a phthalic acid ester, a lactone, a hydroxy carboxylate ester, an ester of a cyclohexane dicarboxylic acid, an ester of a cyclopentane dicarboxylic acid, a lactam, N-pyrolidone, a glycol, and any combination thereof, optionally wherein the solvent is selected from the group consisting of heavy aromatic naphtha, a heavy aromatic naphtha blend, a carboxylic acid, ethyl caprylate, octyl acetate, 2-ethlyhexyl acetate, a dialkyl phthalate, a dialkyl 1,2-cyclohexyldicarboxylate, and any combination thereof, optionally wherein the solvent is selected from the group consisting of heavy aromatic naphtha, toluene, xylene, alkyl benzenes, butyl glycol, diethylene glycol monobutyl ether, or a combination thereof, optionally wherein the solvent is added downstream of a final reactor and upstream of a knockout drum.

6. The method of claim 5, wherein the solvent excludes methanol and/or wherein the method excludes adding methanol to the medium.

7. The method of claim 6, wherein the antipolymerant is selected from the group consisting of 2,2,6,6-tetramethylpiperidinyl-1-oxyl (TEMPO), 4-hydroxy-2,2,6,6-tetramethylpiperidinyl-1-oxyl (HTMPO), 4-oxo-2,2,6,6-tetramethylpiperidinyl-1-oxyl (OTEMPO), di-tert-butyl nitroxyl, 1-oxyl-2,2,6,6-tetramethyl-4-n-propoxypiperidine, 1-oxyl-2,2,6,6-tetramethyl-4-n-butoxypiperidine, 1-oxyl-2,2,6,6-tetramethyl-4-t-butoxypiperidine, 1-oxyl-2,2,6,6-tetramethyl-4-s-butoxypiperidine, 1-oxyl-2,2,6,6-tetramethyl-4-(2-methoxyethoxy)piperidine, 1-oxyl-2,2,6,6-tetramethyl-4-(2-methoxyethoxyacetoxy)piperidine, 1-oxyl-2,2,6,6-tetramethylpiperidin-4-yl stearate, 1-oxyl-2,2,6,6-tetramethylpiperidin-4-yl acetate, 1-oxyl-2,2,6,6-tetramethylpiperidin-4-yl butyrate, 1-oxyl-2,2,6,6-tetramethylpiperidin-4-yl 2-ethylhexanoate, 1-oxyl-2,2,6,6-tetramethylpiperidin-4-yl octanoate, 1-oxyl-2,2,6,6-tetramethylpiperidin-4-yl laurate, 1-oxyl-2,2,6,6-tetramethylpiperidin-4-yl benzoate, 1-oxyl-2,2,6,6-tetramethylpiperidin-4-yl 4-tert-butylbenzoate, 1-oxyl-2,2,6,6-tetramethyl-4-allyloxy-piperidine, 1-oxyl-2,2,6,6-tetramethyl-4-acetamidopiperidine, 1-oxyl-2,2,6,6-tetramethyl-4-(N-butylformamido)piperidine, N-(1-oxyl-2,2,6,6-tetramethylpiperidin-4-yl)-caprolactam, N-(1-oxyl-2,2,6,6-tetramethylpiperidin-4-yl)-dodecylsuccinimide, 1-oxyl-2,2,6,6-tetramethyl-4-(2,3-dihydroxypropoxy)piperidine, 1-oxyl-2,2,6,6-tetramethyl-4-(2-hydroxyl-4-oxapentoxy)piperidine, bis(1-oxyl-2,2,6,6-tetramethylpiperidin-4-yl) succinate, bis(1-oxyl-2,2,6,6-tetramethylpiperidin-4-yl) adipate, bis(1-oxyl-2,2,6,6-tetramethylpiperidin-4-yl) sebacate, bis(1-oxyl-2,2,6,6-tetramethylpiperidin-4-yl) n-butylmalonate, bis(1-oxyl-2,2,6,6-tetramethylpiperidin-4-yl) phthalate, bis(1-oxyl-2,2,6,6-tetramethylpiperidin-4-yl) isophthalate, bis(1-oxyl-2,2,6,6-tetramethylpiperidin-4-yl) terephthalate, bis(1-oxyl-2,2,6,6-tetramethylpiperidin-4-yl) hexahydroterephthalate, N,N′-bis(1-oxyl-2,2,6,6-tetramethylpiperidin-4-yl) adipamide, 2,4,6-tris-[N-butyl-N-(1-oxyl-2,266-tetramethylpiperidin-4-yl)]-s-triazine, 2,4,6-tris-[N-(1-oxyl-2,2,6,6-tetramethylpiperidin-4-yl)]-s-triazine, 4,4′-ethylenebis(1-oxyl-2,2,6,6-tetramethylpiperazin-3-one), a quinone methide, and any combination thereof.

8. The method of claim 1, wherein the antipolymerant is selected from the group consisting of a nitroxide-containing compound comprising a piperidinol group of the following formula: 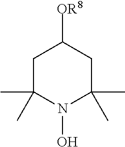
wherein R⁸ is selected from H, a C₁-C₂₂ alkyl group, a C₁-C₂₂ aryl group, a C₁-C₂₂ aryl alkyl group, or a C₁-C₂₂ alkyl aryl group.

9. The method of claim 1, wherein the antipolymerant is selected from the group consisting of a nitroxide-containing compound comprising a piperidinoxy group of the following formula: 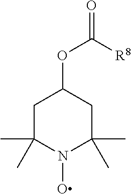
wherein R⁸ is selected from H, a C₁-C₂₂ alkyl group, a C₁-C₂₂ aryl group, a C₁-C₂₂ aryl alkyl group, or a C₁-C₂₂ alkyl aryl group.

10. The method of claim 1, wherein the antipolymerant is selected from the group consisting of a nitroxide-containing compound comprising a piperidinol group of the following formula: 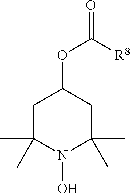
wherein R⁸ is selected from H, a C₁-C₂₂ alkyl group, a C₁-C₂₂ aryl group, a C₁-C₂₂ aryl alkyl group, or a C₁-C₂₂ alkyl aryl group.

11. The method of claim 1, wherein the dispersant is selected from the group consisting of an imidazoline, a fatty acid hydroxyalkylamine ester, a polymer comprising an α-olefin, a polymer comprising maleic anhydride, a polymer comprising an α-olefin and a maleic anhydride in which one or more of the maleic anhydride residues are reacted with a primary amine, a styrene sulfonate polymer, a lignosulfonate metal salt, a naphthalene sulfonate formaldehyde condensate polymer, a polyethoxylated p-alkylphenonol carbonyl condensate polymer, an acrylate polymer, and any combination thereof.

12. The method of claim 1, wherein the antifoulant composition further comprises an antioxidant.

13. The method of claim 1, wherein the medium comprises ethylene and/or propylene.

14. An antifoulant composition, comprising:
a solvent, an antipolymerant, and a dispersant, wherein the solvent is selected from the group consisting of a carboxylic acid ester solvent, a cyclic ester, a phthalic acid ester, a lactone, a hydroxy carboxylate ester, an ester of a cyclohexane dicarboxylic acid, an ester of a cyclopentane dicarboxylic acid, a lactam, N-pyrolidone, a glycol, and any combination thereof.

15. The antifoulant composition of claim 14, wherein the solvent is selected from the group consisting of ethyl caprylate, octyl acetate, dimethyl phthalate, 2-ethlyhexyl acetate, and any combination thereof.

16. The antifoulant composition of claim 14, wherein the solvent excludes methanol and/or wherein the antifoulant composition excludes methanol.

17. The antifoulant composition of claim 14, wherein the antifoulant composition comprises ethylene and/or propylene.

18. The antifoulant composition of claim 14, wherein the antipolymerant is selected from the group consisting of 2,2,6,6-tetramethylpiperidinyl-1-oxyl (TEMPO), 4-hydroxy-2,2,6,6-tetramethylpiperidinyl-1-oxyl (HTMPO), 4-oxo-2,2,6,6-tetramethylpiperidinyl-1-oxyl (OTEMPO), di-tert-butyl nitroxyl, 1-oxyl-2,2,6,6-tetramethyl-4-n-propoxypiperidine, 1-oxyl-2,2,6,6-tetramethyl-4-n-butoxypiperidine, 1-oxyl-2,2,6,6-tetramethyl-4-t-butoxypiperidine, 1-oxyl-2,2,6,6-tetramethyl-4-s-butoxypiperidine, 1-oxyl-2,2,6,6-tetramethyl-4-(2-methoxyethoxy)piperidine, 1-oxyl-2,2,6,6-tetramethyl-4-(2-methoxyethoxyacetoxy)piperidine, 1-oxyl-2,2,6,6-tetramethylpiperidin-4-yl stearate, 1-oxyl-2,2,6,6-tetramethylpiperidin-4-yl acetate, 1-oxyl-2,2,6,6-tetramethylpiperidin-4-yl butyrate, 1-oxyl-2,2,6,6-tetramethylpiperidin-4-yl 2-ethylhexanoate, 1-oxyl-2,2,6,6-tetramethylpiperidin-4-yl octanoate, 1-oxyl-2,2,6,6-tetramethylpiperidin-4-yl laurate, 1-oxyl-2,2,6,6-tetramethylpiperidin-4-yl benzoate, 1-oxyl-2,2,6,6-tetramethylpiperidin-4-yl 4-tert-butylbenzoate, 1-oxyl-2,2,6,6-tetramethyl-4-allyloxy-piperidine, 1-oxyl-2,2,6,6-tetramethyl-4-acetamidopiperidine, 1-oxyl-2,2,6,6-tetramethyl-4-(N-butylformamido)piperidine, N-(1-oxyl-2,2,6,6-tetramethylpiperidin-4-yl)-caprolactam, N-(1-oxyl-2,2,6,6-tetramethylpiperidin-4-yl)-dodecylsuccinimide, 1-oxyl-2,2,6,6-tetramethyl-4-(2,3-dihydroxypropoxy)piperidine, 1-oxyl-2,2,6,6-tetramethyl-4-(2-hydroxyl-4-oxapentoxy)piperidine, bis(1-oxyl-2,2,6,6-tetramethylpiperidin-4-yl) succinate, bis(1-oxyl-2,2,6,6-tetramethylpiperidin-4-yl) adipate, bis(1-oxyl-2,2,6,6-tetramethylpiperidin-4-yl) sebacate, bis(1-oxyl-2,2,6,6-tetramethylpiperidin-4-yl) n-butylmalonate, bis(1-oxyl-2,2,6,6-tetramethylpiperidin-4-yl) phthalate, bis(1-oxyl-2,2,6,6-tetramethylpiperidin-4-yl) isophthalate, bis(1-oxyl-2,2,6,6-tetramethylpiperidin-4-yl) terephthalate, bis(1-oxyl-2,2,6,6-tetramethylpiperidin-4-yl) hexahydroterephthalate, N,N′-bis(1-oxyl-2,2,6,6-tetramethylpiperidin-4-yl) adipamide, 2,4,6-tris-[N-butyl-N-(1-oxyl-2,266-tetramethylpiperidin-4-yl)]-s-triazine, 2,4,6-tris-[N-(1-oxyl-2,2,6,6-tetramethylpiperidin-4-yl)]-s-triazine, 4,4′-ethylenebis(1-oxyl-2,2,6,6-tetramethylpiperazin-3-one), a quinone methide, and any combination thereof.

19. The antifoulant composition of claim 14, wherein the antipolymerant is selected from the group consisting of a nitroxide-containing compound comprising a piperidinol group of the following formula: 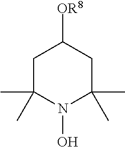
wherein R⁸ is selected from H, a C₁-C₂₂ alkyl group, a C₁-C₂₂ aryl group, a C₁-C₂₂ aryl alkyl group, or a C₁-C₂₂ alkyl aryl group, or
wherein the antipolymerant is selected from the group consisting of a nitroxide-containing compound comprising a piperidinoxy group of the following formula: 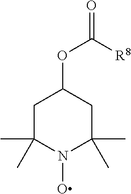
wherein R⁸ is selected from H, a C₁-C₂₂ alkyl group, a C₁-C₂₂ aryl group, a C₁-C₂₂ aryl alkyl group, or a C₁-C₂₂ alkyl aryl group, or
wherein the antipolymerant is selected from the group consisting of a nitroxide-containing compound comprising a piperidinol group of the following formula: 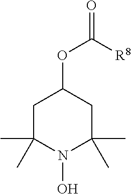
wherein R⁸ is selected from H, a C₁-C₂₂ alkyl group, a C₁-C₂₂ aryl group, a C₁-C₂₂ aryl alkyl group, or a C₁-C₂₂ alkyl aryl group.

20. The antifoulant composition of claim 14, wherein the dispersant is selected from the group consisting of an imidazoline, a fatty acid hydroxyalkylamine ester, a polymer comprising an α-olefin, a polymer comprising maleic anhydride, a polymer comprising an α-olefin and a maleic anhydride in which one or more of the maleic anhydride residues are reacted with a primary amine, a styrene sulfonate polymer, a lignosulfonate metal salt, a naphthalene sulfonate formaldehyde condensate polymer, a polyethoxylated p-alkylphenonol carbonyl condensate polymer, an acrylate polymer, and any combination thereof.
